(19) United States
(12) Patent Application Publication
(10) Pub. No.: US 2019/0295251 A1
(43) Pub. Date: Sep. 26, 2019

(54) INFORMATION PROCESSING APPARATUS AND INFORMATION PROCESSING METHOD

(71) Applicant: CANON KABUSHIKI KAISHA, Tokyo (JP)

(72) Inventor: Kouichi Kato, Yokohama-shi (JP)

(21) Appl. No.: 16/352,700

(22) Filed: Mar. 13, 2019

(30) Foreign Application Priority Data
Mar. 20, 2018 (JP) 2018-052415

(51) Int. Cl. G06T 7/00; G06F 17/18; A61B 8/08; A61B 5/00

(52) U.S. Cl. CPC G06T 7/0012; G06F 17/18; G06T 2207/30101; A61B 5/0059; A61B 5/0095; A61B 8/5207

(57) ABSTRACT

An information processing apparatus includes an acquisition unit, a reception unit, a selection unit, and a processing unit. The acquisition unit acquires a medical image captured by imaging a subject. The reception unit receives specification of a pixel in the acquired medical image. The selection unit selects a predetermined number of pixels in the acquired medical image by searching from the specified pixel. The processing unit performs statistic processing regarding the specified pixel with use of the specified pixel and the selected predetermined number of pixels.

### BACKGROUND

### Field of the Invention

The present disclosure relates to an information processing apparatus and an information processing method.

### Description of the Related Art

Conventionally, there has been a method for setting a region of interest inside an object visualized in a medical image. For example, Japanese Patent Application Laid-Open No. 5-208001 discusses a method by which an image display apparatus sets a region of interest based on a reference point specified from a user (a region growing method). More specifically, the image display apparatus sets one pixel specified from the user as the reference point. Next, the image display apparatus determines a range of values of pixels to be set as the region of interest (for example, an upper limit value and a lower limit value) based on a value of the pixel set as the reference point. The image display apparatus sets pixels having values contained in the determined range as the region of interest.

Meanwhile, the user may check a value of an arbitrary pixel contained in the object visualized in the medical image. In such a case, there is used such a function that, according to specification of the arbitrary pixel contained in the object by the user, an information processing apparatus displays the value of the specified pixel on a display unit such as a display.

However, the pixel specified by the user may be affected by noise or an artifact generated when the object is imaged. This leads to a necessity of comparing a distribution of values of pixels located around the specified pixel and the value of the specified pixel and determining whether the value of the specified pixel is an outlier.

The pixels located around the specified pixel should be collected to make this comparison, but employment of the method discussed in Japanese Patent Application Laid-Open No. 5-208001 causes instances where all pixels having values contained in the range from the upper limit value to the lower limit value are undesirably extracted. More specifically, even a pixel located at a position far away from a position of the specified pixel is undesirably extracted, and it is difficult to extract only the pixels located around the specified pixel according to the method. Thus, even the value of the pixel located at the position far away from the position of the specified pixel is undesirably contained in the distribution of the values of the extracted pixels. Therefore, the distribution of the values of the pixels extracted in this manner is not appropriate as data to be compared with the specified pixel. Further, this method also involves instances where the user needs to set an appropriate upper limit value and lower limit value.

### SUMMARY

The present disclosure is directed to providing an information processing apparatus and an information processing method capable of appropriately performing statistic processing regarding the specified pixel.

Not only the above-described aspect, but also bringing about advantageous effects derived from each configuration that will be indicated in an exemplary embodiment for implementing the present disclosure that will be described below and unable to be acquired by the conventional technique can also be positioned as one of other objects in the present disclosure.

According to an aspect of the present disclosure, an information processing apparatus includes an acquisition unit configured to acquire a medical image captured by imaging a subject, a reception unit configured to receive specification of a pixel in the acquired medical image, a selection unit configured to select a predetermined number of pixels in the acquired medical image by searching from the specified pixel, and a processing unit configured to perform statistic processing regarding the specified pixel with use of the specified pixel and the selected predetermined number of pixels.

Further features will become apparent from the following description of exemplary embodiments with reference to the attached drawings.

### BRIEF DESCRIPTION OF THE DRAWINGS

FIG. 1 illustrates one example of a hardware configuration and a functional configuration of an information processing apparatus.

FIG. 2 illustrates one example of a flow of processing according to an exemplary embodiment.

FIG. 3 illustrates one example of a two-dimensional image.

FIG. 4 illustrates one example of an imaged object.

FIGS. 5A and 5B illustrate one example when a search is conducted in upward, downward, leftward, and rightward directions from a specified pixel and a region containing the specified pixel.

FIG. 6 illustrates one example of a result of searching for a pixel having the closest value to a value of the specified pixel in the upward, downward, leftward, and rightward directions.

FIG. 7 illustrates one example of a result of statistic processing.

FIG. 8 illustrates one example when the search is conducted in upper left, lower left, upper right, and lower right directions from the specified pixel.

FIG. 9 illustrates one example of a result of searching for a pixel having the closest value to a value of a pixel searched last in the upward, downward, leftward, and rightward directions.

FIG. 10 illustrates one example of a result of searching for a pixel having the closest value to a mean value of pixels contained in the region containing the specified pixel and the searched pixel in the upward, downward, leftward, and rightward directions.

### DESCRIPTION OF THE EMBODIMENTS

In the following description, an exemplary embodiment will be described in detail with reference to the drawings. The exemplary embodiment that will be described below is merely one example, and the present disclosure shall not be limited thereto.

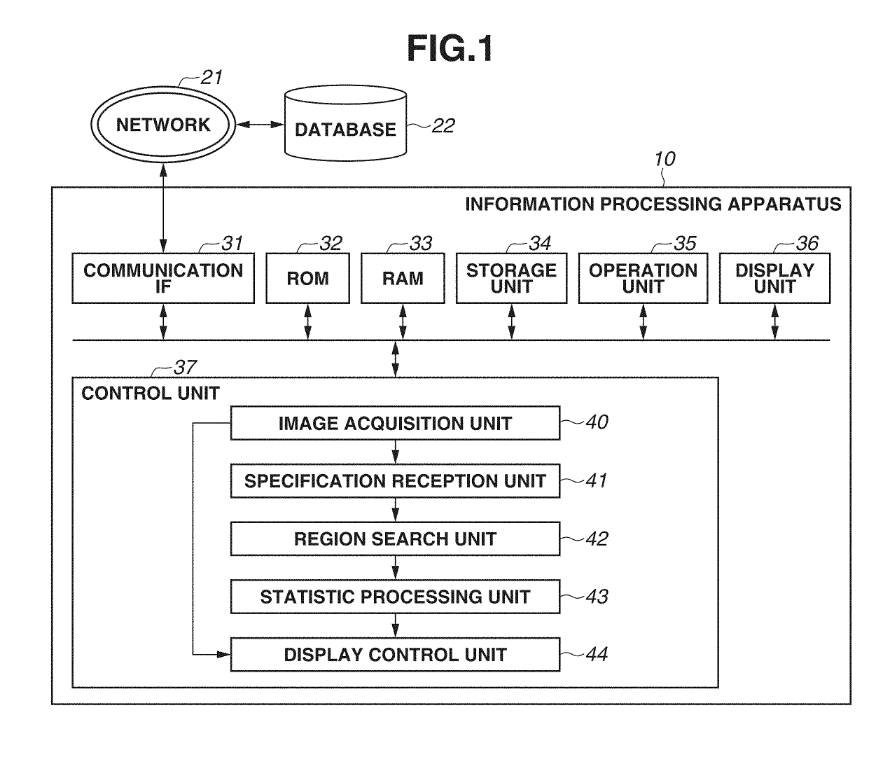

In the following description, a first exemplary embodiment will be described. FIG. 1 illustrates an overall configuration of an information processing system according to the present exemplary embodiment. The information processing system includes an information processing apparatus 10 and a database 22, and these apparatuses are communicably connected to each other via a network 21, which is a communication unit. In the present exemplary embodiment, the communication unit is embodied by a local area network (LAN) 21, but may be a wide area network (WAN). Further, a connection method of the communication unit may be a wired connection or may be a wireless connection.

The database 22 stores a plurality of medical images regarding a plurality of patients and supplementary information about them therein. The medical image refers to two-dimensional or three-dimensional image data acquired by imaging a subject with use of an image diagnosis apparatus (a modality) such as a computed tomography (CT) apparatus, a magnetic resonance imaging (MRI) apparatus, an ultrasonographic apparatus, and an X-ray apparatus. Further, the medical image may be an image based on an acoustic wave emitted from the subject irradiated with light from a light source of a photoacoustic imaging apparatus, or an image indicating a distribution of an oxygen saturation. In the present exemplary embodiment, the information processing system will be described using an example that the medical image is two-dimensional image data. Then, the database 22 is also equipped with functions of, for example, reading out the information stored in the database 22, displaying a list of medical images, displaying a thumbnail, and searching for information. The information processing apparatus 10 acquires various kinds of information stored in the database 22 via the network 21.

The information processing apparatus 10 includes a communication interface (IF) 31, a read only memory (ROM) 32, a random access memory (RAM) 33, a storage unit 34, an operation unit 35, a display unit 36, and a control unit 37.

The communication IF 31 is realized by a LAN card or the like, and is in charge of communication between an external apparatus (for example, the database 22) and the information processing apparatus 10 via the network 21. The ROM 32 is realized by a nonvolatile memory or the like, and stores various kinds of programs therein. The RAM 33 is realized by a volatile memory or the like, and temporarily stores various kinds of information therein. The storage unit 34 is realized by a hard disk drive (HDD) or the like, and stores various kinds of information therein. The operation unit 35 is realized by a keyboard, a mouse, and/or the like, and inputs an instruction from a user to the apparatus. The display unit 36 is realized by a display or the like, and displays various kinds of information for a user (for example, a medical doctor).

The control unit 37 is realized by a central processing unit (CPU) or the like, and comprehensively controls processing in the information processing apparatus 10. The control unit 37 includes an image acquisition unit 40, a specification reception unit 41, a region search unit 42, a statistic processing unit 43, and a display control unit 44, as a functional configuration thereof.

The image acquisition unit 40 acquires the medical image from the database 22 via the communication IF 31 and the network 21 according to an operation of the user which is input by the operation unit 35. In other words, the image acquisition unit 40 corresponds to one example of an acquisition unit configured to acquire a medical image captured by imaging a subject. Then, the image acquisition unit 40 outputs the acquired medical image to the specification reception unit 41 and the display control unit 44.

The specification reception unit 41 receives specification from the user directed to an arbitrary pixel in the medical image displayed on the display unit 36 by the display control unit 44. In other words, the specification reception unit 41 corresponds to one example of a reception unit configured to receive specification of a pixel in the acquired medical image. Then, the specification reception unit 41 outputs information usable to identify the pixel for which the specification is received (for example, coordinates of the pixel or an identification (ID) of the pixel) to the region search unit 42.

The region search unit 42 selects a predetermined number of pixels located around the specified pixel by searching the acquired medical image, placing a starting point of the search at the specified pixel. In other words, the region search unit 42 corresponds to one example of a selection unit configured to select a predetermined number of pixels in the acquired medical image by searching from the specified pixel. Further, the region search unit 42 sets a region of interest that contains the specified pixel and the selected predetermined number of pixels. Then, the region search unit 42 outputs information usable to identify each of the pixels contained in the set region of interest to the statistic processing unit 43.

The statistic processing unit 43 performs statistic processing regarding the specified pixel with use of a value (a pixel value) of each of the pixels contained in the region of interest. In other words, the statistic processing unit 43 corresponds to one example of a processing unit configured to perform statistic processing regarding the specified pixel with use of the specified pixel and the selected predetermined number of pixels. Then, the statistic processing unit 43 outputs a result of the statistic processing to the display control unit 44. The value of the pixel in the present exemplary embodiment refers to a value held by the pixel, and indicates a luminance value, a concentration value, a signal value, an oxygen saturation value, or the like. The same also applies to a voxel value, which will be described below.

The display control unit 44 causes the result of the statistic processing to be displayed on the display unit 36. The result of the statistic processing may be displayed on the display unit 36 together with the medical image.

At least a part of the individual units included in the control unit 37 may be realized as an independent device. Alternatively, each of them may be realized as software that achieves the function. In this case, the software that achieves the function may run on a server via a network, such as a cloud. In the present exemplary embodiment, the individual units are each realized by software under a local environment.

Further, the configuration of the information processing system illustrated in FIG. 1 is merely one example. For example, the storage unit 34 of the information processing apparatus 10 may have the functions of the database 22 and store the plurality of medical images and the supplementary information about them therein.

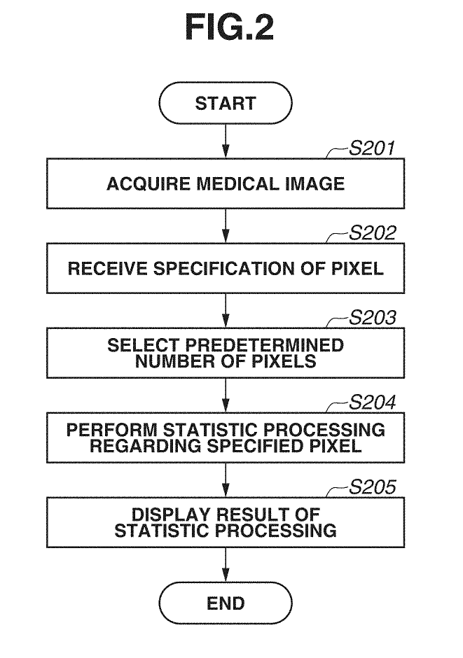

Next, a processing procedure performed by the information processing apparatus 10 according to the present exemplary embodiment will be described in detail with reference to FIG. 2. In the following description, the information processing system will be described using an example that an image acquired by the photoacoustic imaging apparatus is used as the medical image, but the implementation of the present disclosure shall not be limited thereto.

The medical image acquired by the photoacoustic imaging apparatus according to the present exemplary embodiment is a concept including any image derived from a photoacoustic wave generated from the light irradiation. The medical image includes image data indicating a spatial distribution of at least one of a generated sound pressure (an initial sound pressure) of the photoacoustic wave, a density of optical absorption energy, an optical absorption coefficient, information regarding a concentration of a substance forming the subject, and the like. The information regarding the concentration of the substance is a concentration of oxyhemoglobin, a concentration of deoxyhemoglobin, a total concentration of hemoglobin, the oxygen saturation, or the like. The total concentration of hemoglobin refers to a sum of the concentration of oxyhemoglobin and the concentration of deoxyhemoglobin. The oxygen saturation refers to a ratio of oxyhemoglobin to a total amount of hemoglobin. The medical image is not limited to the image indicating the spatial distribution, and may be, for example, an image indicating a numerical value. For example, the medical image is a concept including any image indicating information derived from a photoacoustic signal, such as the photoacoustic signal itself (RAW data), an average concentration of the substance forming the subject, a pixel value at a specific position in the spatial distribution, and a statistic value (a mean value, a median value, or the like) of pixel values in the spatial distribution. For example, a numerical value of the average concentration of the substance forming the subject may be displayed on the display unit 36 as the medical image.

### <Acquire Medical Image>

In step S201, the image acquisition unit 40 acquires the medical image stored in the database 22 via the network 21. In the case where the medical image is stored in the storage unit 34, the image acquisition unit 40 may acquire the medical image from the storage unit 34. Then, the display control unit 44 causes the acquired medical image to be displayed on the display unit 36. The term “acquire” used herein includes, for example, downloading the medical image from the database 22, reading out the medical image from the RAM 33 or the storage unit 34, and storing the medical image in variables.

### <Receive Specification of Pixel>

In step S202, the specification reception unit 41 receives specification of one pixel in the displayed medical image. The user specifies a pixel in a desired tissue visualized in the medical image via the operation unit 35. The specification reception unit 41 receives this specification and stores the information usable to identify the specified pixel (for example, the coordinates or the ID of the specified pixel). Hereinafter, the pixel for which the specification is received from the user will be referred to as the specified pixel. The display control unit 44 may highlight the specified pixel by setting a color to the specified pixel, drawing a thick line as a borderline of the specified pixel, or blinking the specified pixel, although this is not essential processing. Highlighting the specified pixel in such a manner allows the user to easily distinguish the specified pixel from other pixels.

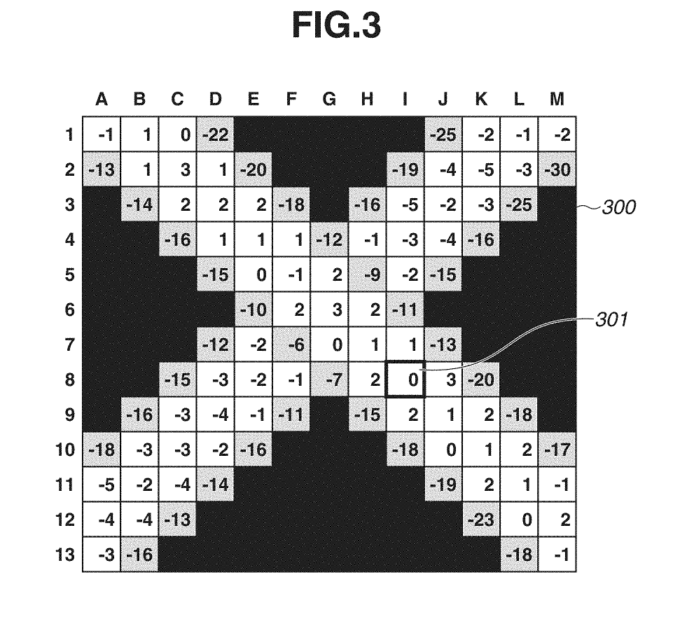

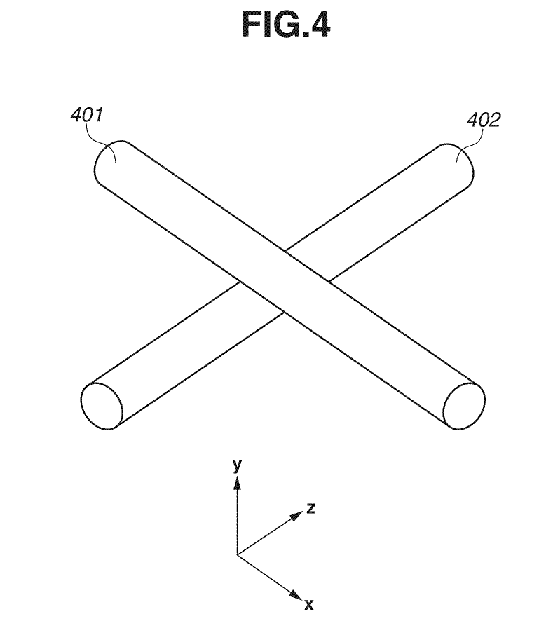

FIG. 3 illustrates one example of the medical image acquired in step S201. Especially, an image 300 indicates a part of a tomographic image acquired by slicing three-dimensional image data (volume data) acquired from the photoacoustic imaging. The image 300 indicates such a situation that a second blood vessel 402 intersects below a first blood vessel 401 as illustrated in FIG. 4. Since blood flows in the internal cavity of a blood vessel, the oxygen saturation is higher therein than in a blood vessel wall. Therefore, in FIG. 3, pixels corresponding to the internal cavity of the blood vessel have higher values than values of pixels corresponding to the blood vessel wall. Further, a pixel labeled 301 is the specified pixel. For the description of the present exemplary embodiment, a value calculated by subtracting a value of the specified pixel 301 from a value of each of the pixels is noted at each of the pixels in the image 300. However, the note of this value is omitted at pixels having low values (blacked pixels). Further, alphabets and numbers are assigned to a horizontal axis (columns) and a vertical axis (rows) of the image 300, respectively. For example, the specified pixel 301 is located at an eighth row in a column I (I8).

### <Select Predetermined Number of Pixels>

In step S203, the region search unit 42 selects a predetermined number of pixels in the medical image by searching from the specified pixel. The predetermined number is set according to the instruction from the user. For example, in a case of a narrow blood vessel having a width of approximately 3 to 5 pixels, it is preferable that approximately 10 is set as the predetermined number. Alternatively, a number that allows the search to be conducted in a range spreading over a radius of approximately 1 mm around the specified pixel may be set as the predetermined number. Further alternatively, the region search unit 42 may estimate a size of the object visualized in the medical image and determine the predetermined number according to the size. In any case, it is desirable that the number of pixels that allows a neighborhood around the specified pixel to be searched is set as the predetermined number.

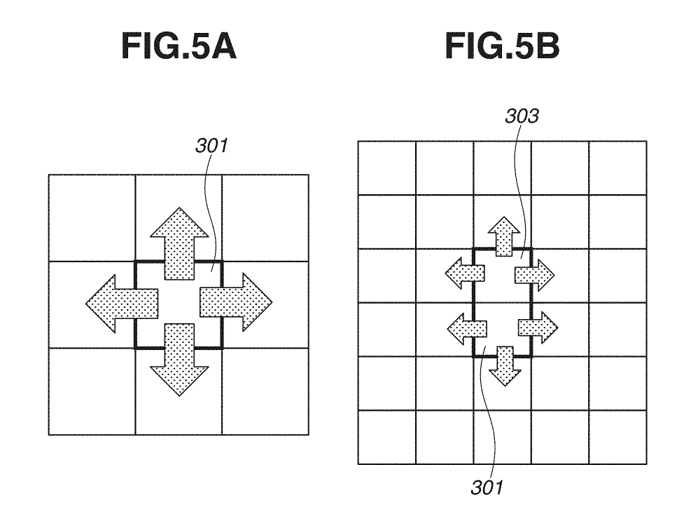

Various methods can be employed as a method for the search, but the search will be described to be conducted by a method that searches pixels adjacent to the specified pixel or the region of interest in four directions (upward, downward, leftward, and rightward directions) as illustrated in FIGS. 5A and 5B in the present exemplary embodiment.

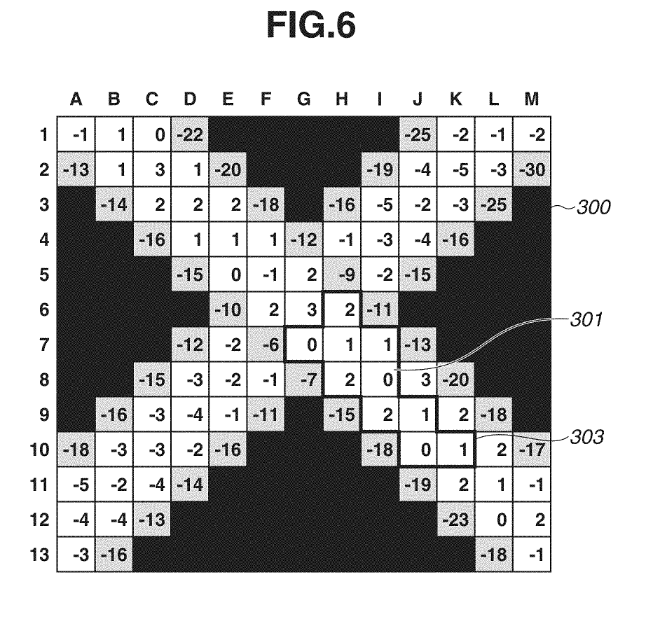

(1) First, the region search unit 42 selects a pixel having the closest value to the value of the specified pixel 301 among pixels adjacent to the specified pixel 301 in the four directions that are the upward, downward, leftward, and rightward directions. In the example illustrated in FIG. 3, a pixel I7 is selected. Based on this selection, the region search unit 42 sets the region of interest that contains the specified pixel 301 and the selected pixel I7. (2) Next, the region search unit 42 selects a pixel having the closest value to the value of the specified pixel 301 among pixels adjacent to the region of interest in the four directions that are the upward, downward, leftward, and rightward directions. For example, if one pixel has been selected, the region search unit 42 searches pixels located in directions indicated by six arrows illustrated in FIG. 5B. In the example illustrated in FIG. 3, a pixel H7 is selected. Then, the region search unit 42 adds the newly selected pixel H7 to the region of interest. If there is a plurality of pixels having the closest value to the value of the specified pixel 301, the region search unit 42 selects a pixel located at a shortest distance from the specified pixel 301. If the plurality of pixels is located at equal distances from the specified pixel 301, the region search unit 42 selects the pixel according to a priority order among the upper, lower, left, and right sides that is set by the user in advance. (3) Then, the region search unit 42 repeatedly carries out (2) and ends the search when the predetermined number of pixels is selected. FIG. 6 illustrates a result of searching by the above-described search method from the specified pixel 301. A region 303 illustrated in FIG. 6 is the region of interest that contains the specified pixel 301 and the selected predetermined number of pixels. The region search unit 42 selects the predetermined number of pixels in the medical image by searching from the specified pixel 301 in this manner.

Further, the display control unit 44 may highlight the region of interest by setting a color to the region of interest, drawing a thick line as a borderline of the region of interest, or blinking the region of interest, although this is not essential processing. Highlighting the region of interest in such a manner allows the user to easily distinguish the region of interest from other pixels from each other.

Searching for the predetermined number of pixels in the four directions brings about the following advantageous effects. For example, supposing that the user has intended to specify a pixel corresponding to the internal cavity of the blood vessel but mistakenly specified a pixel corresponding to the blood vessel wall (pixels in gray in FIG. 3), the following consequence may arise in this case. Searching from the specified pixel according to the conventional region growing method unintentionally causes the search range to also contain pixels located in diagonal directions, thereby undesirably resulting in extraction of the entire blood vessel wall. This extraction fails to lead to a rejection of the value of the pixel corresponding to the blood vessel wall in statistic processing that will be described below. According to the present exemplary embodiment, the pixels corresponding to the internal cavity of the blood vessel that are located around the specified pixel will also be selected even when the pixel corresponding to the blood vessel wall is specified. This selection allows the statistic processing unit 43 to determine that the value of the specified pixel corresponding to the blood vessel wall is an outlier with respect to the values of the pixels located around the specified pixel.

Further, another conceivable configuration is that the region search unit 42 simply selects eight pixels (H7, H8, H9, 17, 19, J7, J8, and J9) surrounding the specified pixel (I8). However, in this case, even the pixel corresponding to the blood vessel wall is unintentionally selected despite the fact that the internal cavity of the blood vessel is specified, so that the value of the specified pixel may be unintentionally rejected in the statistic processing that will be described below. Therefore, it is necessary to search the predetermined number of pixels in the four directions in the above-described manner.

<Perform Statistic Processing regarding Specified Pixel>

In step S204, the statistic processing unit 43 performs the statistic processing regarding the specified pixel with use of the value of each of the pixels contained in the region of interest. More specifically, the statistic processing unit 43 determines whether the value of the specified pixel deviates from the value of each of the pixels located around the specified pixel, i.e., the value of the specified pixel is the outlier. In the present exemplary embodiment, the statistic processing will be described with use of Thompson's rejection test. First, the statistic processing unit 43 calculates a t value with use of the following equations (1) and (2).

$$\begin{matrix}τ = \frac{\vert M - a \vert}{\sqrt{\frac{\sum {( x - M )}^{2}}{N}}} & Equation ( 1 ) \\ t = \frac{τ \sqrt{N - 2}}{\sqrt{N - 1 - {τ}^{2}}} & Equation ( 2 )\end{matrix}$$

### M: Mean Value

### a: Value of Specified Pixel

x: Value of Specified Pixel and Value of Selected pixel

### N: The Number of Collected Pixels+1

### Degree of Freedom: N−2

### t: t Value

The statistic processing unit 43 compares the t value calculated with use of the equation (2) and a t distribution table, thereby conducting a significant difference test at a desired level. For example, the statistic processing unit 43 conducts a two-sided test at a level of 0.1%. As a result, the statistic processing unit 43 can determine whether the specified pixel is the outlier. In the present exemplary embodiment, the statistic processing unit 43 employs Thompsons's rejection test, but may employ Smirnoff-Grubbs' rejection test or may employ the rejection test developed by Doctor Motosaburo Masuyama. Alternatively, the statistic processing unit 43 may determine that the specified pixel is the outlier if the value of the specified pixel does not fall within a range defined by a mean value of the values of the pixels in the region of interest ±a standard deviation××a constant A as the detection of the outlier. Normally, a value in a range from 2 to 3 is used as the constant A, but the constant A can be changed freely by the user.

### <Display Result of Statistic Processing>

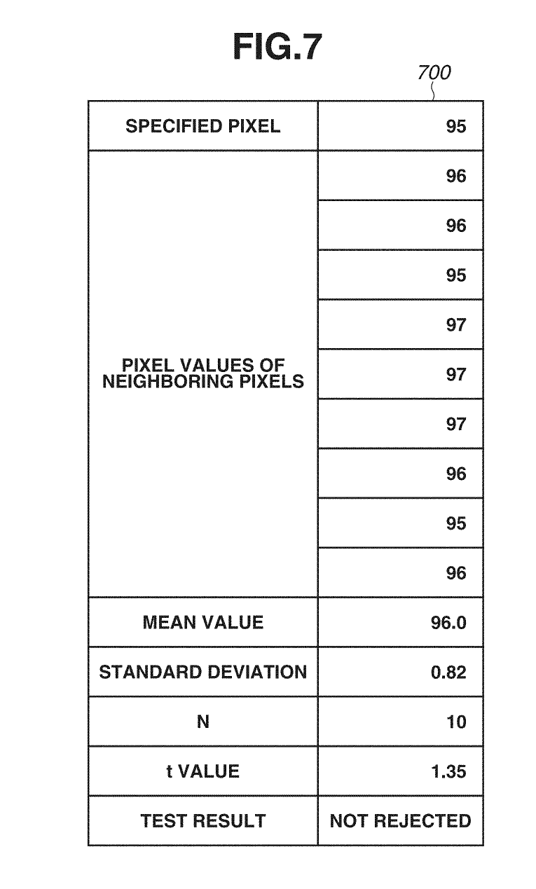

In step S205, the display control unit 44 causes the result of the statistic processing to be displayed on the display unit 36. The display control unit 44 may cause the result of the statistic processing to be displayed on the display unit 36 together with the medical image at this time. FIG. 7 illustrates one example of the result of the statistic processing. A table 700 indicates the value of each of the pixels contained in the region of interest, the mean value of the pixels contained in the region of interest, the standard deviation, the number of pixels, the t value, and the result of the determination from the rejection test. The result of the statistic processing is not limited thereto, and may contain a result of a determination from another rejection test. Further, the display control unit 44 may cause an alert to be displayed on the display unit 36 to prompt the user to change the specified pixel when the value of the specified pixel is determined to be the outlier.

According to the present exemplary embodiment, it is possible to select pixels in a necessary range, thereby appropriately performing the statistic processing.

### First Modification Example

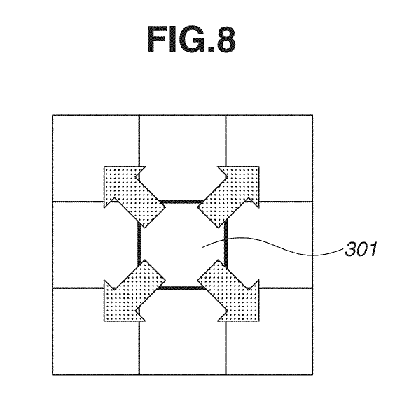

The search has been described referring to the method that searches from the specified pixel in the four directions that are the upward, downward, leftward, and rightward directions in the present exemplary embodiment, but may be conducted by a method that searches from the specified pixel in four diagonal directions. More specifically, as illustrated in FIG. 8, the search may be conducted from the specified pixel 301 in four directions that are upper left, lower left, upper right, and lower right directions. For example, supposing that the blood vessel extends vertically or horizontally and a pixel corresponding to the blood vessel wall is specified, the following consequence may arise in this case. In this case, searching from the specified pixel in the four directions that are the upward, downward, leftward, and rightward directions may unintentionally result in extraction of only the blood vessel wall since the blood vessel extends vertically or horizontally. In such a case, therefore, the intended result can be maintained by searching from the specified pixel in the four diagonal directions.

Whether to search in the four directions that are the upward, downward, leftward, and rightward directions or search in the four diagonal directions may be selected by the user prior to the search or may be automatically determined based on detection of the shape of the blood vessel.

### Second Modification Example

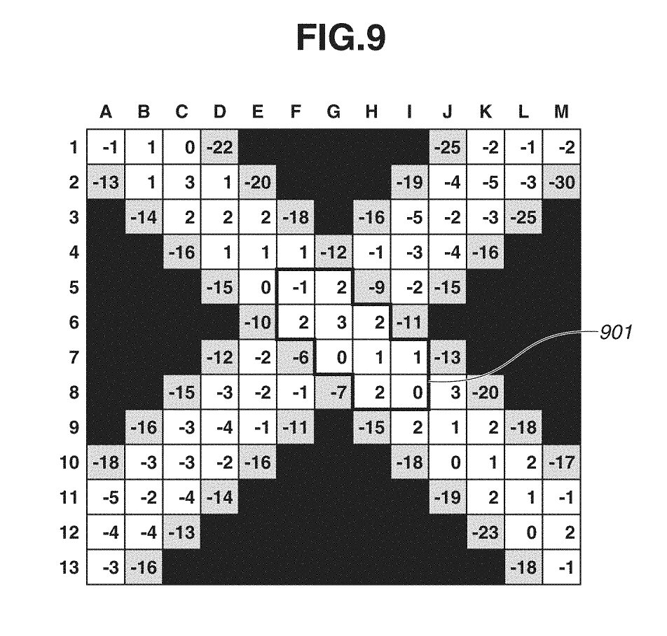

Further, the selection of the pixel has been described referring to the method that selects the pixel having the closest value to the value of the specified pixel in the present exemplary embodiment, but the information processing apparatus may select a pixel having the closest value to a value of a predetermined pixel contained in the region of interest. For example, the pixel may be selected by a method that selects a pixel having the closest value to a value of a pixel selected last as a next selected pixel. This method will be described with reference to FIG. 9. When the specified pixel is 18 similarly to FIG. 3, the pixel 17 is selected first by searching in a similar manner to the above-described exemplary embodiment. Then, a pixel having the closest value to the value of the pixel 17 is selected from the pixels adjacent in the four directions to the region of interest. A region of interest 901 illustrated in FIG. 9 is set by repeating this selection. Selecting the pixel in this manner enables the search to be conducted with directionality. Especially, the search can proceed in a constant direction at the time of the search the internal cavity of the blood vessel visualized with two to three pixels. It can also be understood that the search proceeds toward the upper left side in FIG. 9. The information processing apparatus may select a pixel having the closest value to a value of a pixel selected immediately before the last selected pixel instead of the value of the last selected pixel.

### Third Modification Example

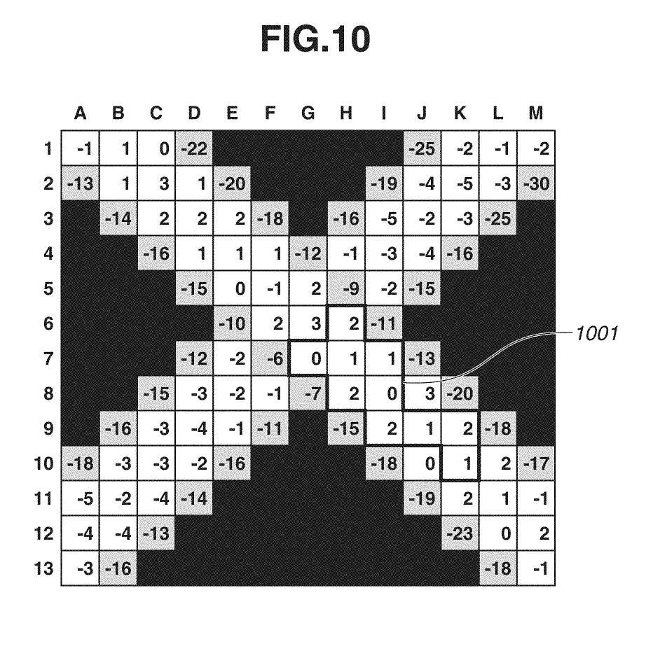

Further, the selection of the pixel has been described referring to the method that selects the pixel having the closest value to the value of the specified pixel in the present exemplary embodiment, but the pixel may be selected by a method that selects a pixel having the closest value to the mean value of the pixels contained in the region of interest. This method will be described with reference to FIG. 10. When the specified pixel is 18 similarly to FIG. 3, the pixel 17 is selected first by searching in a similar manner to the above-described exemplary embodiment. Then, the mean value is calculated with respect to the pixels (17 and 18) contained in the region of interest, and a pixel having the closest value to this mean value is selected from the pixels adjacent to the region of interest in the four directions. In this case, because the mean value is 0.5, a pixel H7 is selected. A region of interest 1001 illustrated in FIG. 10 is set by repeating this selection. Selecting the pixel in this manner can reduce such a possibility that a region outside the internal cavity of the blood vessel is searched when the specified pixel is the pixel corresponding to the internal cavity of the blood vessel.

### Fourth Exemplary Modification

Further, the pixel selection method may be switched based on a selection from the user among the pixel selection method according to the present exemplary embodiment, the pixel selection method according to the second exemplary modification, and the pixel selection method according to the third exemplary modification. It is desirable to employ an appropriate selection method according to the shape of the object visualized in the medical image and/or the value of the pixel. Further, the selection method may be switched every time one or more pixels is(are) selected.

### Fifth Exemplary Modification

Further, the medical image is the two-dimensional image data in the present exemplary embodiment, but the medical image may be three-dimensional image data. In other words, the medical image may be volume data. In this case, similar advantageous effects can be achieved by replacing the term “pixel” described in the present exemplary embodiment and each of the exemplary modifications with the term “voxel”. Further, as the search direction in this case, the search is conducted from the specified pixel in six directions that are upward, downward, leftward, rightward, frontward, and backward directions.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2018-052415, filed Mar. 20, 2018, which is hereby incorporated by reference herein in its entirety.

What is claimed is:

1. An information processing apparatus comprising:
an acquisition unit configured to acquire a medical image captured by imaging a subject;
a reception unit configured to receive specification of a pixel in the acquired medical image;
a selection unit configured to select a predetermined number of pixels in the acquired medical image by searching from the specified pixel; and
a processing unit configured to perform statistic processing regarding the specified pixel with use of the specified pixel and the selected predetermined number of pixels.

2. The information processing apparatus according to claim 1, wherein the processing unit determines whether a value of the specified pixel is an outlier with respect to the selected predetermined number of pixels by performing the statistic processing.

3. The information processing apparatus according to claim 1, wherein the selection unit searches a pixel adjacent to the specified pixel and selects a pixel having a closest value to a value of the specified pixel.

4. The information processing apparatus according to claim 1, wherein the selection unit searches a pixel adjacent to a region containing the specified pixel and the pixels selected by the search and selects a pixel having a closest value to a value of the specified pixel.

5. The information processing apparatus according to claim 1, wherein the selection unit searches a pixel adjacent to a region containing the specified pixel and the pixels selected by the search and selects a pixel having a closest value to a value of a predetermined pixel contained in the region.

6. The information processing apparatus according to claim 1, wherein the selection unit searches a pixel adjacent to a region containing the specified pixel and the pixels selected by the search and selects a pixel having a closest value to a mean value of the pixels contained in the region.

7. The information processing apparatus according to claim 1, wherein the selection unit searches adjacent pixels in four directions from the specified pixel.

8. The information processing apparatus according to claim 4, wherein the selection unit searches adjacent pixels in four directions from the region.

9. An information processing apparatus comprising:
an acquisition unit configured to acquire a medical image captured by imaging a subject;
a reception unit configured to receive specification of a voxel in the acquired medical image;
a selection unit configured to select a predetermined number of voxels in the acquired medical image by searching from the specified voxel; and
a processing unit configured to perform statistic processing regarding the specified voxel with use of the specified voxel and the selected predetermined number of voxels.

10. The information processing apparatus according to claim 9, wherein the processing unit determines whether a value of the specified voxel is an outlier with respect to the selected predetermined number of voxels by performing the statistic processing.

11. The information processing apparatus according to claim 9, wherein the selection unit searches a voxel adjacent to the specified voxel and selects a voxel having a closest value to a value of the specified voxel.

12. The information processing apparatus according to claim 9, wherein the selection unit searches a voxel adjacent to a region containing the specified voxel and the voxels selected by the search and selects a voxel having a closest value to a value of the specified voxel.

13. The information processing apparatus according to claim 9, wherein the selection unit searches a voxel adjacent to a region containing the specified voxel and the voxels selected by the search and selects a voxel having a closest value to a value of a predetermined voxel contained in the region.

14. The information processing apparatus according to claim 9, wherein the selection unit searches a voxel adjacent to a region containing the specified voxel and the voxels selected by the search and selects a voxel having a closest value to a mean value of the voxels contained in the region.

15. The information processing apparatus according to claim 9, wherein the selection unit searches adjacent voxels in six directions from the specified voxel.

16. The information processing apparatus according to claim 12, wherein the selection unit searches adjacent voxels in six directions from the region.

17. The information processing apparatus according to claim 1, wherein the statistic processing is a rejection test.

18. The information processing apparatus according to claim 1, wherein the subject is a blood vessel.

19. The information processing apparatus according to claim 1, wherein the medical image is an image based on an acoustic wave emitted from the subject irradiated with light from a light source.

20. The information processing apparatus according to claim 1, wherein the medical image is an image indicating a distribution of an oxygen saturation.

21. An information processing method comprising:
acquiring a medical image captured by imaging a subject;
receiving specification of a pixel in the acquired medical image;
selecting a predetermined number of pixels in the acquired medical image by searching from the specified pixel; and
performing statistic processing regarding the specified pixel with use of the specified pixel and the selected predetermined number of pixels.

22. An information processing method comprising:
acquiring a medical image captured by imaging a subject;
receiving specification of a voxel in the acquired medical image;
selecting a predetermined number of voxels in the acquired medical image by searching from the specified voxel; and
performing statistic processing regarding the specified voxel with use of the specified voxel and the selected predetermined number of voxels.
